(19) United States
(12) Patent Application Publication
(10) Pub. No.: US 2024/0311692 A1
(43) Pub. Date: Sep. 19, 2024

(54) MODEL GENERATION DEVICE, PREDICTION DEVICE, MODEL GENERATION METHOD, PREDICTION METHOD, AND RESIN COMPOSITION MANUFACTURING SYSTEM

(71) Applicant: TOKUYAMA CORPORATION, Yamaguchi (JP)

(72) Inventors: Minami TAI, Yamaguchi (JP); Shunsuke IMAI, Yamaguchi (JP); Go HAMASAKA, Yamaguchi (JP)

(21) Appl. No.: 18/571,042

(22) PCT Filed: Jun. 24, 2022
(86) PCT No.: PCT/JP2022/025344

§ 371 Date: Dec. 15, 2023

(30) Foreign Application Priority Data
Jul. 20, 2021 (JP) 2021-119567

(51) Int. Cl. G06N 20/00

(52) U.S. Cl. CPC G06N 20/00

(57) ABSTRACT

In order to find a required characteristic satisfying condition regarding a resin composition more efficiently than a conventional technology, a model generation device (100) is configured such that: a first machine learning section (21) generates, on the basis of first input data (110) and second input data (120) that makes a pair with the first input data (110), a first prediction model (MODEL 1) that predicts unknown resin composition characteristic data from at least one selected from the group consisting of (i) given inorganic filling material characteristic data, (ii) given resin characteristic data, (iii) given inorganic filling material proportion data and (iv) given resin proportion data; and a second machine learning section (22) generates, on the basis of the first prediction model (MODEL 1), a second prediction model (MODEL 2) that predicts at least one selected from the group consisting of (i) predicted inorganic filling material characteristic data, (ii) predicted resin characteristic data, (iii) predicted inorganic filling material proportion data and (iv) predicted resin proportion data, the at least one satisfying given resin composition characteristic data.

### TECHNICAL FIELD

An aspect of the present invention relates to a model generation device that generates a prediction model for predicting a required characteristic satisfying condition (described later) regarding a resin composition.

### BACKGROUND ART

In order to obtain a resin composition having a desired required characteristic (performance), it is necessary to find a condition (e.g., makeup) of a resin composition that satisfies the required characteristic. However, conventionally, composition examination by carrying out many trials and errors on an experimental basis was necessary in order to find out the condition. For example, Patent Literature 1 discloses that in order to find suitable makeup of an epoxy resin composition satisfying a required characteristic that “bleed can be inhibited”, it was necessary to carry out a number of experiments.

### CITATION LIST

### Patent Literature

### [Patent Literature 1]

Japanese Patent Application Publication Tokukai No. 2015-105304

### SUMMARY OF INVENTION

### Technical Problem

An object of an aspect of the present invention is to find at least one selected from items (i) to (iv) below more efficiently than a conventional technology, the items (i) to (iv) satisfying a required characteristic of a resin composition that contains at least one inorganic filling material and at least one resin: (i) a characteristic of the inorganic filling material; (ii) a characteristic of the resin; (iii) a mixing ratio of the inorganic filling material; and (iv) a mixing ratio of the resin (in the present specification, collectively referred to as “required characteristic satisfying condition”).

### Solution to Problem

In order to solve the above problem, a model generation device in accordance with an aspect of the present invention is
a model generation device configured to generate a prediction model for predicting at least one selected from the group consisting of the following (i) to (iv), the at least one satisfying a required characteristic of a resin composition which contains at least one inorganic filling material and at least one resin: (i) a characteristic of the inorganic filling material; (ii) a characteristic of the resin; (iii) a mixing ratio of the inorganic filling material; and (iv) a mixing ratio of the resin, the model generation device including:
a first input data acquisition section configured to acquire first input data that is input data including at least one selected from the group consisting of (i) inorganic filling material characteristic input data indicating the characteristic of the inorganic filling material, (ii) resin characteristic input data indicating the characteristic of the resin, (iii) inorganic filling material proportion input data related to the mixing ratio of each of a plurality of the inorganic filling materials in the resin composition obtained by mixing the plurality of the inorganic filling materials in the resin and (iv) resin proportion input data related to the mixing ratio of the resin;
a second input data acquisition section configured to acquire, as second input data that makes a pair with the first input data, resin composition characteristic input data indicating a characteristic of the resin composition;
a first machine learning section configured to generate, on the basis of the first input data and the second input data, a first prediction model that predicts unknown resin composition characteristic data from at least one selected from the group consisting of (i) given inorganic filling material characteristic data, (ii) given resin characteristic data, (iii) given inorganic filling material proportion data and (iv) given resin proportion data; and
a second machine learning section configured to generate, on the basis of the first prediction model, a second prediction model that predicts at least one selected from the group consisting of (i) predicted inorganic filling material characteristic data, (ii) predicted resin characteristic data, (iii) predicted inorganic filling material proportion data and (iv) predicted resin proportion data, the at least one satisfying given resin composition characteristic data.

Further, a prediction device in accordance with the present invention is
a prediction device configured to predict at least one selected from the group consisting of the following (i) to (iv), the at least one satisfying a required characteristic of a resin composition which contains at least one inorganic filling material and at least one resin: (i) a characteristic of the inorganic filling material; (ii) a characteristic of the resin; (iii) a mixing ratio of the inorganic filling material; and (iv) a mixing ratio of the resin, wherein:
first input data is acquired in advance, the first input data being input data including at least one selected from the group consisting of (i) inorganic filling material characteristic input data indicating the characteristic of the inorganic filling material, (ii) resin characteristic input data indicating the characteristic of the resin, (iii) inorganic filling material proportion input data related to the mixing ratio of each of a plurality of the inorganic filling materials in the resin composition obtained by mixing the plurality of the inorganic filling materials in the resin and (iv) resin proportion input data related to the mixing ratio of the resin;
resin composition characteristic input data is acquired in advance, as second input data that makes a pair with the first input data, the resin composition characteristic input data indicating a characteristic of the
a first prediction model is generated in advance, on the basis of the first input data and the second input data, the first prediction model predicting unknown resin composition characteristic data from at least one selected from the group consisting of (i) given inorganic filling material characteristic data, (ii) given resin characteristic data, (iii) given inorganic filling material proportion data and (iv) given resin proportion data;
a second prediction model is generated in advance, on the basis of the first prediction model, the second prediction model predicting at least one selected from the group consisting of (i) predicted inorganic filling material characteristic data, (ii) predicted resin characteristic data, (iii) predicted inorganic filling material proportion data and (iv) predicted resin proportion data, the at least one satisfying given resin composition characteristic data; and
the prediction device includes:
   a third input data acquisition section configured to acquire, as third input data, resin composition required characteristic data indicating the required characteristic of the resin composition; and
   a recommended data deriving section configured to derive recommended data by inputting the resin composition required characteristic data to the second prediction model, the recommended data including at least one selected from the group consisting of (i) recommended inorganic filling material characteristic data indicating the characteristic of the inorganic filling material that satisfies the resin composition required characteristic data, (ii) recommended resin characteristic data indicating the characteristic of the resin that satisfies the resin composition required characteristic data, (iii) recommended inorganic filling material proportion data indicating a proportion of the inorganic filling material that satisfies the resin composition required characteristic data and (iv) recommended resin proportion data indicating a proportion of the resin that satisfies the resin composition required characteristic data.

Further, a model generation method in accordance with an aspect of the present invention is
a method for generating a prediction model for predicting at least one selected from the group consisting of the following (i) to (iv), the at least one satisfying a required characteristic of a resin composition which contains at least one inorganic filling material and at least one resin: (i) a characteristic of the inorganic filling material; (ii) a characteristic of the resin; (iii) a mixing ratio of the inorganic filling material; and (iv) a mixing ratio of the resin,
the method including:
a first input data acquisition step of acquiring first input data that is input data including at least one selected from the group consisting of (i) inorganic filling material characteristic input data indicating the characteristic of the inorganic filling material, (ii) resin characteristic input data indicating the characteristic of the resin, (iii) inorganic filling material proportion input data related to the mixing ratio of each of a plurality of the inorganic filling materials in the resin composition obtained by mixing the plurality of the inorganic filling materials in the resin and (iv) resin proportion input data related to the mixing ratio of the resin;
a second input data acquisition step of acquiring, as second input data that makes a pair with the first input data, resin composition characteristic input data indicating a characteristic of the resin composition;
a first machine learning step of generating, on the basis of the first input data and the second input data, a first prediction model that predicts unknown resin composition characteristic data from at least one selected from the group consisting of (i) given inorganic filling material characteristic data, (ii) given resin characteristic data, (iii) given inorganic filling material proportion data and (iv) given resin proportion data; and
a second machine learning step of generating, on the basis of the first prediction model, a second prediction model that predicts at least one selected from the group consisting of (i) predicted inorganic filling material characteristic data, (ii) predicted resin characteristic data, (iii) predicted inorganic filling material proportion data and (iv) predicted resin proportion data, the at least one satisfying given resin composition characteristic data.

Further, a prediction method in accordance with an aspect of the present invention is
a method for predicting at least one selected from the group consisting of the following (i) to (iv), the at least one satisfying a required characteristic of a resin composition which contains at least one inorganic filling material and at least one resin: (i) a characteristic of the inorganic filling material; (ii) a characteristic of the resin; (iii) a mixing ratio of the inorganic filling material; and (iv) a mixing ratio of the resin, wherein:
first input data is acquired in advance, the first input data being input data including at least one selected from the group consisting of (i) inorganic filling material characteristic input data indicating the characteristic of the inorganic filling material, (ii) resin characteristic input data indicating the characteristic of the resin, (iii) inorganic filling material proportion input data related to the mixing ratio of each of a plurality of the inorganic filling materials in the resin composition obtained by mixing the plurality of the inorganic filling materials in the resin and (iv) resin proportion input data related to the mixing ratio of the resin;
resin composition characteristic input data is acquired in advance, as second input data that makes a pair with the first input data, the resin composition characteristic input data indicating a characteristic of the resin composition;
a first prediction model is generated in advance, on the basis of the first input data and the second input data, the first prediction model predicting unknown resin composition characteristic data from at least one selected from the group consisting of (i) given inorganic filling material characteristic data, (ii) given resin characteristic data, (iii) given inorganic filling material proportion data and (iv) given resin proportion data;
a second prediction model is generated in advance, on the basis of the first prediction model, the second prediction model predicting at least one selected from the group consisting of (i) predicted inorganic filling material characteristic data, (ii) predicted resin characteristic data, (iii) predicted inorganic filling material proportion data and (iv) predicted resin proportion data, the at least one satisfying given resin composition characteristic data; and
the method includes:
   a third input data acquisition step of acquiring, as third input data, resin composition required characteristic data indicating the required characteristic of the resin composition; and
   a recommended data deriving step of deriving recommended data by inputting the resin composition required characteristic data to the second prediction model, the recommended data including at least one selected from the group consisting of (i) recommended inorganic filling material characteristic data indicating the characteristic of the inorganic filling material that satisfies the resin composition required characteristic data, (ii) recommended resin characteristic data indicating the characteristic of the resin that satisfies the resin composition required characteristic data, (iii) recommended inorganic filling material proportion data indicating a proportion of the inorganic filling material that satisfies the resin composition required characteristic data and (iv) recommended resin proportion data indicating a proportion of the resin that satisfies the resin composition required characteristic data.

Further, a resin composition production system in accordance with an aspect of the present invention includes:
a model generation device configured to generate a prediction model for predicting at least one selected from the group consisting of the following (i) to (iv), the at least one satisfying a required characteristic of a resin composition which contains at least one inorganic filling material and at least one resin: (i) a characteristic of the inorganic filling material; (ii) a characteristic of the resin; (iii) a mixing ratio of the inorganic filling material; and (iv) a mixing ratio of the resin; and
a prediction device configured to predict, with use of the prediction model generated by the model generation device, at least one selected from the group consisting of the following (i) to (iv), the at least one satisfying the required characteristic of the resin composition: (i) the characteristic of the inorganic filling material; (ii) the characteristic of the resin; (iii) the mixing ratio of the inorganic filling material; and (iv) the mixing ratio of the resin,
the model generation device including:
   a first input data acquisition section configured to acquire first input data that is input data including at least one selected from the group consisting of (i) inorganic filling material characteristic input data indicating the characteristic of the inorganic filling material, (ii) resin characteristic input data indicating the characteristic of the resin, (iii) inorganic filling material proportion input data related to the mixing ratio of each of a plurality of the inorganic filling materials in the resin composition obtained by mixing the plurality of the inorganic filling materials in the resin and (iv) resin proportion input data related to the mixing ratio of the resin;
   a second input data acquisition section configured to acquire, as second input data that makes a pair with the first input data, resin composition characteristic input data indicating a characteristic of the resin composition;
   a first machine learning section configured to generate, on the basis of the first input data and the second input data, a first prediction model that predicts unknown resin composition characteristic data from at least one selected from the group consisting of (i) given inorganic filling material characteristic data, (ii) given resin characteristic data, (iii) given inorganic filling material proportion data and (iv) given resin proportion data; and
      a second machine learning section configured to generate, on the basis of the first prediction model, a second prediction model that predicts at least one selected from the group consisting of (i) predicted inorganic filling material characteristic data, (ii) predicted resin characteristic data, (iii) predicted inorganic filling material proportion data and (iv) predicted resin proportion data, the at least one satisfying given resin composition characteristic data,
the prediction device including:
   a third input data acquisition section configured to acquire, as third input data, resin composition required characteristic data indicating the required characteristic of the resin composition; and
   a recommended data deriving section configured to derive recommended data by inputting the resin composition required characteristic data to the second prediction model, the recommended data including at least one selected from the group consisting of (i) recommended inorganic filling material characteristic data indicating the characteristic of the inorganic filling material that satisfies the resin composition required characteristic data, (ii) recommended resin characteristic data indicating the characteristic of the resin that data, (iii) recommended inorganic filling material proportion data indicating a proportion of the inorganic filling material that satisfies the resin composition required characteristic data and (iv) recommended resin proportion data indicating a proportion of the resin that satisfies the resin composition required characteristic data.

### Advantageous Effects of Invention

An aspect of the present invention makes it possible to find a required characteristic satisfying condition regarding a resin composition, more efficiently than a conventional technology.

### BRIEF DESCRIPTION OF DRAWINGS

FIG. 1 is a block diagram illustrating a configuration of a main part of a resin composition production system according to Embodiment 1.

FIG. 2 is a diagram illustrating an overview of a first prediction model.

FIG. 3 is a diagram illustrating an overview of a second prediction model.

FIG. 4 is a diagram illustrating an overview of a recommended data deriving section.

FIG. 5 is a diagram showing an example of each of inorganic filling material proportion input data and second input data.

FIG. 6 is a diagram illustrating an example of the inorganic filling material characteristic input data.

FIG. 7 is a diagram illustrating an example of deriving an explanatory variable in a first algorithm execution section.

FIG. 8 is a diagram illustrating an example of generation of a first prediction model in a second algorithm execution section.

FIG. 9 is a diagram illustrating an example of input and output in the first prediction model.

FIG. 10 is a diagram showing an operation example in a second prediction model generated by a third algorithm execution section.

### DESCRIPTION OF EMBODIMENTS

### Embodiment 1

The following description will discuss a resin composition production system 1 in accordance with Embodiment 1. For convenience of explanation, constituent elements (components) which have functions identical with those described in Embodiment 1 will be given the same reference numerals as those in Embodiment 1, and descriptions as to such constituent elements will not be repeated. For simplification, descriptions of matters identical to those of a well-known technique will be also omitted as appropriate. Note that each configuration and each numerical value described in the present specification are examples, unless otherwise clearly stated. In the present specification, the expression “X to Y” regarding two numbers x and y means “not less than X and not more than Y”, unless otherwise clearly stated.

### (Overview of Resin Composition Production System 1)

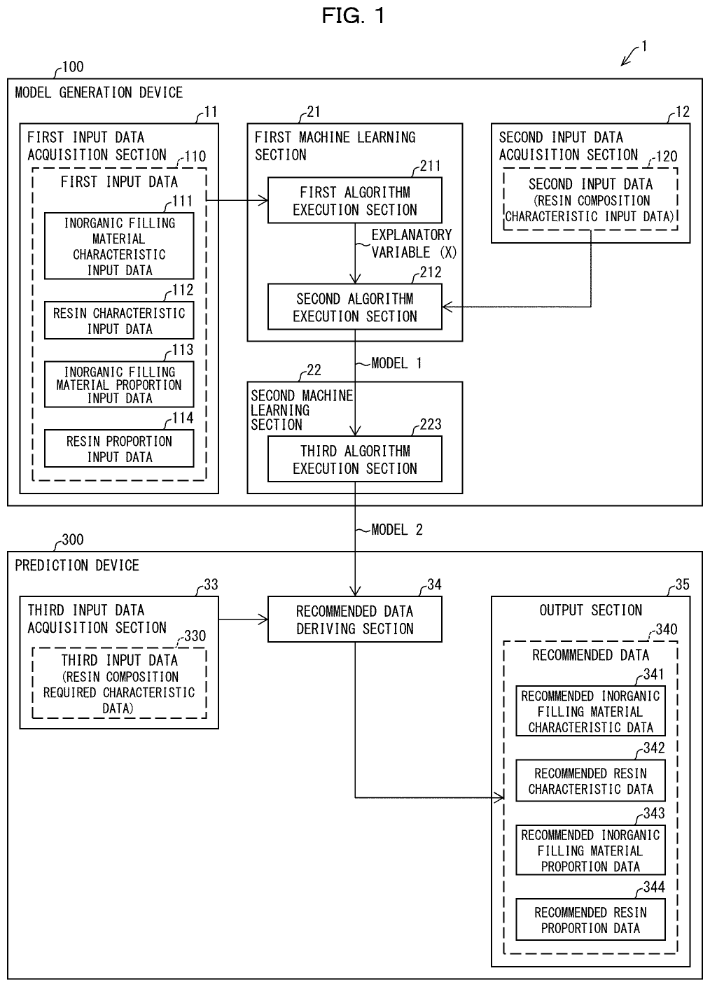

FIG. 1 is a block diagram illustrating a configuration of a main part of a resin composition production system 1. The resin composition production system 1 includes a model generation device 100 and a prediction device 300. As will be described later, the model generation device 100 generates a prediction model for predicting a required characteristic satisfying condition regarding a resin composition which contains at least one inorganic filling material and at least one resin. More specifically the model generation device 100 generates, as the prediction model, a second prediction model (MODEL 2), which will be described later. The prediction device 300 predicts the required characteristic satisfying condition with use of MODEL 2.

In the following description, at least one inorganic filling material is collectively referred to as an inorganic filling material A and at least one resin is collectively referred to as a resin B. Further, a resin composition containing the inorganic filling material A and the resin B is referred to as a resin composition C.

Main examples of the inorganic filling material A include ceramic (e.g., silica, alumina, aluminum nitride, silicon nitride, and boron nitride). The inorganic filling material is also referred to as an inorganic filler. It is possible to change a characteristic of the resin composition C by changing an amount of the inorganic filling material A added to the resin composition C. For example, it is possible to improve an elastic modulus and a linear expansion coefficient of the resin composition C by appropriately setting the amount of the inorganic filling material A added. Embodiment 1 describes, as a major example, a case where the inorganic filling material A is silica.

Main examples of the resin B include any organic polymer compound. The resin composition C contains the inorganic filling material A and the resin B. The resin composition C may further contain another material (e.g.: a curing agent, elastomer, an ion trapping agent, a pigment, a dye, an anti-foaming agent, a stress relaxing agent, a pH adjusting agent, an accelerating agent, a surfactant, and a coupling agent). Main examples of the resin composition C include an adhesive and a sealant.

Note that the “required characteristic satisfying condition” in the present specification is not limited to a “condition that perfectly satisfies a required characteristic of the resin composition C”. Examples of the “required characteristic satisfying condition” in the present specification include a “required characteristic closest to a perfect required characteristic of the resin composition C” and a “required characteristic close in some measure to the perfect required characteristic of the resin composition C”. Therefore, the resin composition production system 1 only needs to be capable of predicting, as the required characteristic satisfying condition, a condition that mostly satisfies the perfect required characteristic of the resin C. For this purpose, for example, as described later, the resin composition production system 1 may predict the required characteristic satisfying condition on the basis of a stochastic mathematical model.

### (Model Generation Device 100)

A process in the resin composition production system 1 is roughly divided into a learning phase (process in the model generation device 100) and a prediction phase (process in the prediction device 300). The prediction phase is also referred to as an inference phase. First, the following will describe the learning phase. The model generation device 100 includes a first input data acquisition section 11, a second input data acquisition section 12, a first machine learning section 21, and a second machine learning section 22.

The first input data acquisition section 11 acquires first input data 110. The first input data 110 includes at least one selected from the group consisting of:
inorganic filling material characteristic input data 111;
resin characteristic input data 112;
inorganic filling material proportion input data 113; and
resin proportion input data 114.

The inorganic filling material characteristic input data 111 indicates a characteristic of the inorganic filling material A. The resin characteristic input data 112 indicates a characteristic of the resin B. The inorganic filling material proportion input data 113 is data related to a mixing ratio of the inorganic filling material A in the resin composition C which is obtained by mixing a plurality of types of the inorganic filling material A in the resin B. For example, the inorganic filling material proportion input data 113 indicates a mixing ratio of the inorganic filling material A. The resin proportion input data 114 is related to a mixing ratio of the resin B in the resin composition C. For example, the resin proportion input data 114 is data indicating a mixing ratio of the resin B.

The first input data acquisition section 11 may be any data acquisition interface. For example, the first input data acquisition section 11 may be an input section configured to receive an input operation of a user. In this case, the first input data acquisition section 11 acquires the first input data 110 inputted by the user. As another example, the first input data acquisition section 11 may acquire the first input data 110 that has been previously stored in a storage section (not shown) in the model generation device 100. As still another example, the first input data acquisition section 11 may communicate with an external device (not shown) outside the model generation device 100, and acquire the first input data 110 from the external device. Examples of the external device include a storage server and a measurement device. These descriptions of the first input data acquisition section 11 apply similarly to the second input data acquisition section 12 described below and a third input data acquisition section 33 described later.

The second input data acquisition section 12 acquires second input data 120. More specifically, the second input data acquisition section 12 acquires, as the second input data 120, resin composition characteristic input data. The resin composition characteristic input data is data indicating the characteristic of the resin composition C. Therefore, it can be said that the second input data 120 is data that makes a pair with the first input data 110.

The first machine learning section 21 acquires the first input data 110 from the first input data acquisition section 11 and the second input data 120 from the second input data acquisition section 12. The first machine learning section 21 generates a first prediction model (hereinafter, referred to as MODEL 1) on the basis of the first input data 110 and the second input data 120.

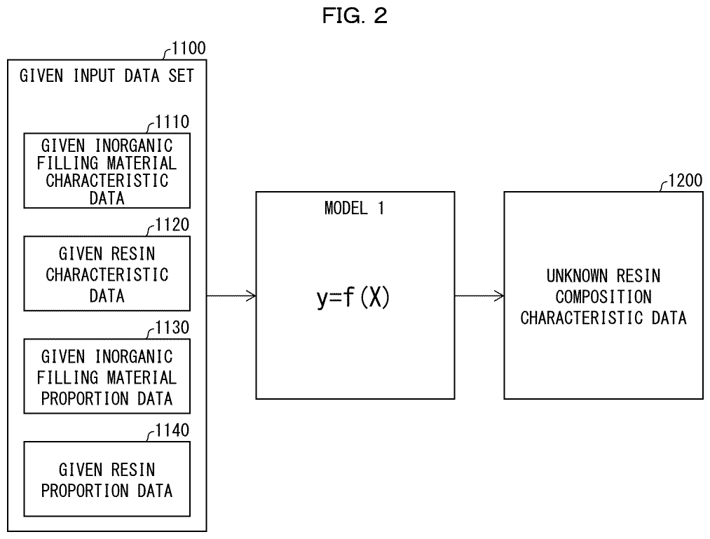

FIG. 2 is a diagram illustrating an overview of MODEL 1. As illustrated in FIG. 2, MODEL 1 is a model (mathematical model) for predicting unknown resin composition characteristic data 1200 from at least one selected from the group consisting of the following:
given inorganic filling material characteristic data 1110;
given resin characteristic data 1120;
given inorganic filling material proportion data 1130; and
given resin proportion data 1140.

In the present specification, a data set including at least one selected from the group consisting of the given inorganic filling material characteristic data 1110, the given resin characteristic data 1120, the given inorganic filling material proportion data 1130, and the given resin proportion data 1140 is referred to as a given input data set 1100.

In an example of FIG. 2, the given input data set 1100 is an example of an explanatory variable (X). Further, the unknown resin composition characteristic data 1200 is an example of an objective variable (y). Note that explanatory variable is also referred to as an independent variable. In contrast, the objective variable is also referred to as a dependent variable or an explained variable. In the example of FIG. 2, MODE 1 can be expressed as a function f indicating a relationship y=f(X). Thus, MODEL 1 is a model for solving a forward problem (a model for deriving y from X).

Note that in the present specification, it is assumed that a type of data included in the given input data set 1100 matches a type of teacher data used for generation of MODEL 1. That is, it is assumed that a data structure of the given input data set 1100 matches a data structure of the teacher data used for generation of MODEL 1. Thus, for example, the data structure of the given input data set 1100 matches a data structure of the first input data 110.

The second machine learning section 22 acquires MODEL 1 from the first machine learning section 21. The second machine learning section 22 generates a second prediction model (hereinafter, referred to as MODEL 2) on the basis of MODEL 1.

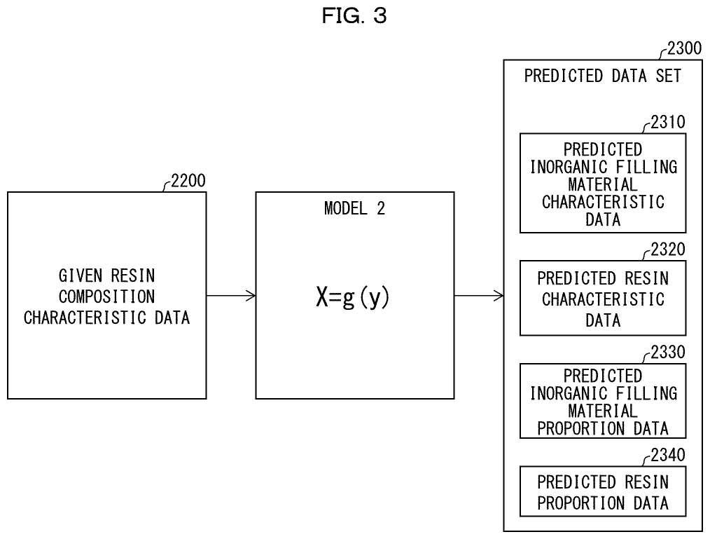

FIG. 3 is a diagram illustrating an overview of MODEL 2. As illustrated in FIG. 3, MODEL 2 is a model for predicting at least one selected from the group consisting of the following pieces of data:
predicted inorganic filling material characteristic data 2310;
predicted resin characteristic data 2320;
predicted inorganic filling material proportion data 2330; and
predicted resin proportion data 2340,
   the at least one satisfying the given resin composition characteristic data 2200. Note that the expression “satisfying the given resin composition characteristic data 2200” in the present specification means “satisfying the required characteristic of the resin composition C indicated by the given resin composition characteristic data 2200”. Further, in the present specification, a data set including at least one selected from the group consisting of the predicted inorganic filling material characteristic data 2310, the predicted resin characteristic data 2320, the predicted inorganic filling material proportion data 2330, and the predicted resin proportion data 2340 is referred to as a predicted data set 2300.

In an example of FIG. 3, the given resin composition characteristic data 2200 is an example of the objective variable (y). Further, the predicted data set 2300 is an example of the explanatory variable (X). In the example of FIG. 3, MODEL 2 can be expressed as a function g indicating the relationship X=g(y). Note that g≈f⁻¹. That is, the function g is an approximate inverse function of the function f. Thus, MODEL 2 is a model for solving an inverse problem (a model for deriving X from y). As described above, MODEL 2 is a model that makes a pair with MODEL 1.

Note that in the present specification, it is assumed that a type of the given resin composition characteristic data 2200 matches a type of teacher data used for generation of MODEL 2. That is, it is assumed that a data structure of the given resin composition characteristic data 2200 matches a data structure of the teacher data used for generation of MODEL 2. Thus, for example, the data structure of the given resin composition characteristic data 2200 matches a data structure of the second input data 120.

In an example of FIG. 1, the first machine learning section 21 has a first algorithm execution section 211 and a second algorithm execution section 212. The first algorithm execution section 211 acquires the first input data 110 from the first input data acquisition section 11. The first algorithm execution section 211 executes a first algorithm for deriving the explanatory variable (X) corresponding to data that indicates the characteristic of the resin composition C and that is obtained from the second input data 120. Examples of the first algorithm will be described later.

The second algorithm execution section 212 acquires the explanatory variable (X) from the first algorithm execution section 211 and also acquires the second input data 120 from the second input data acquisition section 12. The second algorithm execution section 212 executes a second algorithm for generating MODEL 1 on the basis of X and the second input data 120.

The second algorithm may be any algorithm capable of generating MODEL 1 on the basis of X and second input data 120. In other words, the second algorithm may be any algorithm capable of generating a model for solving a forward problem. For example, the second algorithm is at least one selected from the group consisting of:
Gaussian process regression;
support-vector machine;
linear regression;
decision tree;
random forest;
neural network; and
gradient boosting decision tree.

In the example of FIG. 1, the second machine learning section 22 has a third algorithm execution section 223. The third algorithm execution section 223 executes a third algorithm for generating MODEL 2 on the basis of MODEL 1.

The third algorithm may be any algorithm capable of generating MODEL 2 on the basis of MODEL 1. In other words, the third algorithm may be any algorithm capable of generating a model for solving an inverse problem. For example, the third algorithm is at least one selected from the group consisting of:
genetic algorithm;
gradient descent;
grid search; and
Bayesian optimization.

In an aspect of the present invention, the inorganic filling material characteristic input data 111 may be data indicating any characteristic of the inorganic filling material A. For example, the inorganic filling material characteristic input data 111 indicates at least one selected from the group consisting of the following as the characteristic of the inorganic filling material A:
composition formula, crystallinity, specific gravity, bulk specific gravity, particle size distribution, specific surface area, pore volume, zeta potential, specific electric conductivity, dielectric constant, dielectric dissipation factor, refractive index, specific heat, thermal conductivity, linear expansion coefficient, crushing strength, sphericity, aspect ratio, moisture content, carbon content, nitrogen content, surface functional group species, surface functional group content, light absorption wavelength, light absorbance, M-value and solubility parameter of the inorganic filling material A.

Note that the description regarding the inorganic filling material characteristic input data 111 applies similarly to each data corresponding to the inorganic filling material characteristic input data 111.

In an aspect of the present invention, the resin characteristic input data 112 may be data indicating any characteristic of the resin B. For example, the resin characteristic input data 112 indicates at least one selected from the group consisting of the following as the characteristic of the resin B:
composition formula, polymerization degree, molecular weight distribution, stereoregularity, reactive functional group species, reactive functional group content, viscosity, melting point, glass transition temperature, crystallinity, elastic modulus, yield stress, breaking strength, fracture toughness, light absorption wavelength, light absorbance, specific gravity, refractive index, specific electric conductivity, dielectric constant, dielectric dissipation factor, specific heat, thermal conductivity, moisture content and solubility parameter of the resin B.
   Note that the description regarding the resin characteristic input data 112 similarly applies to each data corresponding to the resin characteristic input data 112.

In an aspect of the present invention, the resin composition characteristic input data (second input data 120) may be data indicating any characteristic of the resin composition C. For example, the resin composition characteristic input data indicates at least one selected from the group consisting of the following as the characteristic of the resin composition C:
viscosity, flowability, moldability, adhesiveness, transparency, color tone, strength, water absorption rate, linear expansion coefficient, elastic modulus, yield stress, breaking strength, fracture toughness, specific electric conductivity, dielectric constant, dielectric dissipation factor, thermal conductivity and stability of the resin composition C.

Note that the description regarding the resin composition characteristic input data similarly applies to each data corresponding to the resin composition characteristic input data.

### (Prediction Device 300)

Next, the following will describe the prediction phase. The prediction device 300 includes a third input data acquisition section 33, a recommended data deriving section 34, and an output section 35.

The third input data acquisition section 33 acquires third input data 330. More specifically the third input data acquisition section 33 acquires resin composition required characteristic data as the third input data 330. The resin composition required characteristic data indicates the required characteristic of the resin composition C.

The recommended data deriving section 34 acquires the resin composition required characteristic data (third input data 330) from the third input data acquisition section 33, and also acquires MODEL 2 from the model generation device 100 (more specifically, the second machine learning section 22).

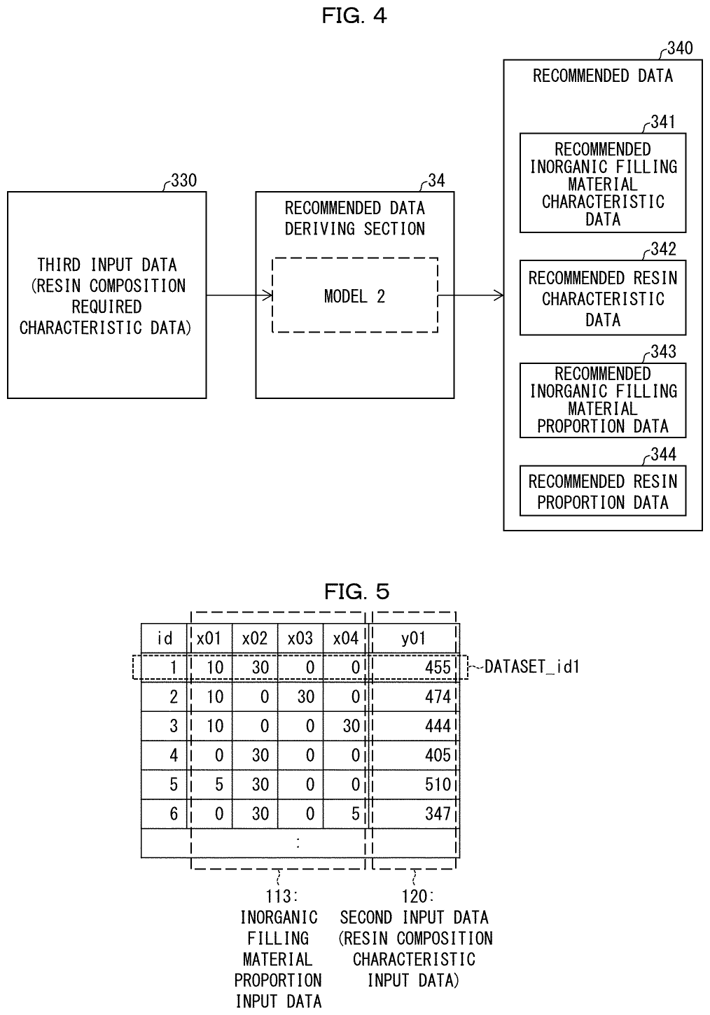

FIG. 4 is a diagram illustrating an overview of the recommended data deriving section 34. As illustrated in FIG. 4, the recommended data deriving section 34 derives recommended data 340 by inputting the resin composition required characteristic data to MODEL 2. The recommended data 340 includes at least one selected from the group consisting of the following:
recommended inorganic filling material characteristic data 341;
recommended resin characteristic data 342;
recommended inorganic filling material proportion data 343; and
recommended resin proportion data 344.

The recommended inorganic filling material characteristic data 341 indicates a characteristic of the inorganic filling material A that satisfies the resin composition required characteristic data. The recommended resin characteristic data 342 indicates a characteristic of the resin B that satisfies the resin composition required characteristic data. The recommended inorganic filling material proportion data 343 indicates a proportion of the inorganic filling material A that satisfies the resin composition required characteristic data. The recommended resin proportion data 344 indicates a proportion of the resin B that satisfies the resin composition required characteristic data. Note that the expression “satisfying the resin composition required characteristic data” in the present specification means “satisfying the required characteristic of the resin composition C indicated by the resin composition required characteristic data”. Specific examples of a process for deriving the recommended data 340 by the recommended data deriving section 34 will be described later.

As described above, the expression “required characteristic satisfying condition” in the present specification is not limited to a “condition that perfectly satisfies the required characteristic of the resin composition C”. Therefore, naturally, the recommended data 340 in the present specification is not limited to the “data that perfectly satisfies the resin composition required characteristic data”. The recommended data 340 in the present specification also includes the “data that mostly satisfies the resin composition required characteristic data”.

Therefore, the recommended inorganic filling material characteristic data 341 in the present specification only needs to indicate a characteristic of the inorganic filling material A that mostly satisfies the resin composition required characteristic data. Similarly, the recommended resin characteristic data 342 only needs to indicate a characteristic of the resin B that mostly satisfies the resin composition required characteristic data. Further, the recommended inorganic filling material proportion data 343 only needs to indicate a proportion of the inorganic filling material A that mostly satisfies the resin composition required characteristic data. Similarly, the recommended resin proportion data 344 only needs to indicate a proportion of the resin B that mostly satisfies the resin composition required characteristic data. Note that the above descriptions regarding the recommended inorganic filling material characteristic data 341, the recommended resin characteristic data 342, the recommended inorganic filling material proportion data 343, and the recommended resin proportion data 344 similarly apply to the predicted inorganic filling material characteristic data 2310, the predicted resin characteristic data 2320, the predicted inorganic filling material proportion data 2330, and the predicted resin proportion data 2340 described above.

The output section 35 acquires the recommended data 340 from the recommended data deriving section 34. The output section 35 outputs the recommended data 340. The output section 35 may be any output interface. For example, the output section 35 may be a display (display device). In this case, the output section 35 can visually present the recommended data 340 to the user by displaying the recommended data 340. In this manner, the output section 35 may output the recommended data 340 in a visual manner. As another example, the output section 35 may transfer the recommended data 340 to the storage section in the model generation device 100. As yet another example, the output section 35 may transfer the recommended data 340 to an external device outside the model generation device 100.

In an aspect of the present invention, the resin composition required characteristic data (third input data 330) may be data indicating any required characteristic of the resin composition C. As is apparent from the description regarding the resin composition characteristic input data described above, for example, the resin composition required characteristic data indicates, as the required characteristic of the resin composition C, at least one selected from the group consisting of
viscosity, flowability, moldability, adhesiveness, transparency, color tone, strength, water absorption rate, linear expansion coefficient, elastic modulus, yield stress, breaking strength, fracture toughness, specific electric conductivity, dielectric constant, dielectric dissipation factor, thermal conductivity and stability of the resin composition C.

### (One Example of Process in Resin Composition Production System 1)

The following will describe an example of the process in the resin composition production system 1. In the following example, a case where five types of the inorganic filling material A and one type of the resin B are used will be described. In the following example, it is assumed that both of the characteristic of the resin B and the mixing ratio of the resin B are set to constant fixed values (fixed conditions).

Therefore, in the following example, the characteristic of the resin B and the mixing ratio of the resin B are each not considered as an explanatory variable. Accordingly, in the following description, the resin characteristic input data 112 and the resin proportion input data 114 are not mentioned. In light of the above, in the following description, the mixing ratio of the inorganic filling material A is also referred to simply as “mixing ratio”. Note that in the following example, the characteristic of the resin composition C is exemplified by a viscosity (unit: Pa·s) of the resin composition C. Further, the viscosity of the resin composition C is also referred to simply as “viscosity”.

### (Example of Each of First Input Data 110 and Second Input Data 120)

FIG. 5 is a diagram illustrating an example of each of the inorganic filling material proportion input data 113 and the second input data 120. The inorganic filling material proportion input data 113 in the example of FIG. 5 is data indicating respective mixing ratios of the five types of inorganic filling materials in percent by weight (wt %). In the following description, the five types of inorganic filling materials are respectively referred to as inorganic filling materials 0 to 4. Further, the mixing ratio of an inorganic filling material i is referred to as x0i. i is an integer satisfying 0≤i≤4. For example, x01 represents a mixing ratio of the inorganic filling material 1.

Here, as would be apparent to a person skilled in the art, the following relation is established:

$$x 00 + x 01 + x 02 + x 03 + x 04 = 100.$$

Therefore, x00 is uniquely determined in accordance with preset x01 to x04 as follows:

$$x 00 = 100 - x 01 - x 02 - x 03 - x 4.$$

Therefore, in the inorganic filling material proportion input data 113 in the example of FIG. 5, only x01 to x04 are set in order to reduce the number of dimensions of the explanatory variable.

In the inorganic filling material proportion input data 113, a plurality of mixing ratio patterns (patterns of a combination of x01 to x04) are set. In FIG. 5, id is an identification number of such a mixing ratio pattern. For example, id=1 indicates a 1st mixing ratio pattern (hereinafter, also referred to as a first mixing ratio pattern). In the first mixing ratio pattern in the example of FIG. 5, “x00=60, x01=10, x02=30, x03=0, and x04=0”. Note that in the following, for example, id=1 is referred to simply as “id1”, as appropriate.

The second input data 120 in the example of FIG. 5 indicates the viscosity (y01) of the resin composition C corresponding to each of the mixing ratio patterns. More specifically, a value of the viscosity actually measured for each mixing ratio pattern is recorded in y01 of the second input data 120. Prior to computer simulation in the present example, the inventors of the present application (hereinafter, simply referred to as “inventors”) produced the resin composition C according to the first mixing ratio pattern described above. Further, when the inventors actually measured the viscosity of the resin composition C, a measured value of 455 was obtained. Accordingly, in the second input data 120 in the example of FIG. 5, the value of y01=455 is set for id1.

In the example of FIG. 5, a data set indicating a corresponding relationship between each mixing ratio pattern and y01 is created for each id. In the example of FIG. 5, a data set corresponding to the jth id (idj) is referred to as DATASET_idj. For example, DATASET_id1 is a data set indicating a corresponding relationship between the first mixing ratio pattern and y01. Hereinafter, a jth mixing ratio pattern is also referred to as a j-th mixing ratio pattern j.

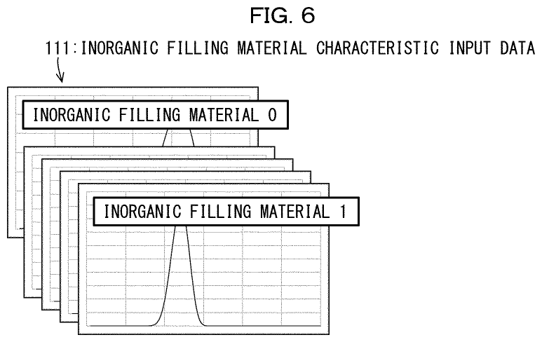

FIG. 6 is a diagram illustrating an example of the inorganic filling material characteristic input data 111. The inorganic filling material characteristic input data 111 in the example of FIG. 6 indicates a particle size distribution of each of the inorganic filling materials 0 to 4.

### (Example of Deriving Explanatory Variable in First Algorithm Execution Section 211)

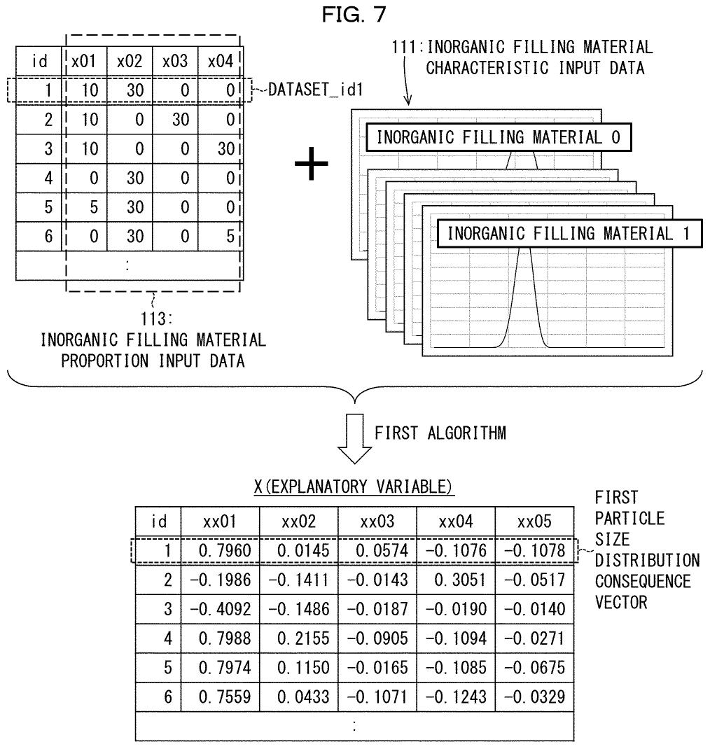

FIG. 7 is a diagram illustrating an example of deriving the explanatory variable in the first algorithm execution section 211. The first algorithm execution section 211 derives the explanatory variable (X) corresponding to data that indicates the characteristic of the resin composition C and that is obtained from the first input data 110. In the present example, the first algorithm execution section 211 derives the explanatory variable corresponding to data that indicates the viscosity of the resin composition C and that is obtained from the inorganic filling material proportion input data 113 and the inorganic filling material characteristic input data 111. As described above, in the present example, the explanatory variable for explaining the viscosity, which is the objective variable, is derived as X.

More specifically, the first algorithm execution section 211 derives X on the basis of the inorganic filling material proportion input data 113 and the inorganic filling material characteristic input data 111 by executing the first algorithm. In the example of FIG. 7, the first machine learning section 21 performs, as the first algorithm, weighted average calculation and principal component analysis.

In the example of FIG. 7, the first machine learning section 21 performs weighted average calculation of the inorganic filling material proportion input data 113 on the basis of the inorganic filling material characteristic input data 111. Subsequently, the first machine learning section 21 performs principal component analysis of the inorganic filling material proportion input data after the weighted average calculation, and derives, as the explanatory variable, a particle size distribution consequence vector (a vector having xx01 to xx05 in FIG. 7 as components). Note that a weighted value in the weighted average calculation may be set by a well-known method on the basis of, for example, the inorganic filling material characteristic input data 111. A dimension to be reduced by the principal component analysis may be set to any dimension. The first machine learning section 21 calculates the particle size distribution consequence vector for each id. Therefore, for example, as illustrated in FIG. 7, the explanatory variable derived by the first machine learning section 21 includes a first particle size distribution consequence vector (particle size distribution consequence vector corresponding to id1) described below.

In the present specification, a particle size distribution consequence vector corresponding to the j-th mixing ratio pattern (in other words, a particle size distribution consequence vector corresponding to idj) is referred to as a j-th particle size distribution consequence vector. The j-th particle size distribution consequence vector is calculated from particle size distributions of the inorganic filling materials 0 to 4 in the resin composition C in a case where the j-th mixing ratio pattern is applied. Therefore, for example, the first particle size distribution consequence vector is calculated from the particle size distributions of the inorganic filling materials 0 to 4 in the resin composition C in a case where the first mixing ratio pattern described above is applied.

### (Example of Generation of MODEL 1 in Second Algorithm Execution Section 212)

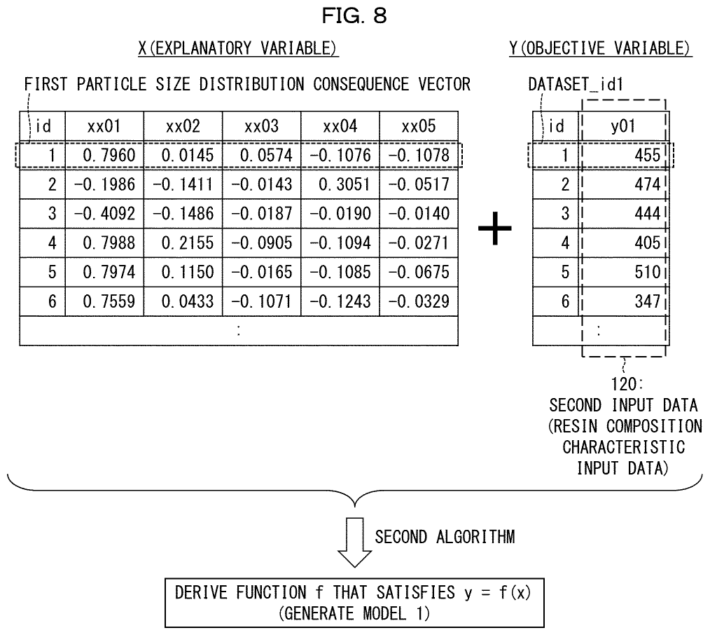

FIG. 8 is a diagram illustrating an example of generation of MODEL 1 in the second algorithm execution section 212. The second algorithm execution section 212 generates MODEL 1 on the basis of (i) the explanatory variable (X) derived by the first algorithm execution section 211 and (ii) the second input data 120. More specifically the second algorithm execution section 212 generates MODEL 1 on the basis of X and the second input data 120, by executing the second algorithm.

In the example of FIG. 8, the second algorithm execution section 212 executes, as the second algorithm, a neural network. More specifically, the second algorithm execution section 212 acquires, for each id, an objective variable corresponding to X from the second input data 120. For example, the second algorithm execution section 212 acquires, as correct data of the objective variable (y) corresponding to X for id1, the viscosity (y01) indicated in DATASET_id1. The second algorithm execution section 212 derives a function f that satisfies the relationship between X and y for each id by executing the neural network with the use of the correct data. The second algorithm execution section 212 thus generates MODEL 1 as the function f that indicates the relationship of y=f(X).

The following will describe, as an example, a case in which the second algorithm execution section 212 generates MODEL 1 by carrying out ensemble learning by a bagging method. Therefore, MODEL 1 in the present example includes a plurality of neural nets (weak learning devices) having different hyperparameters. MODEL 1 in the present example is thus generated as a strong learning device in which a plurality of weak learning devices are integrated.

### (Example of Input and Output of MODEL 1)

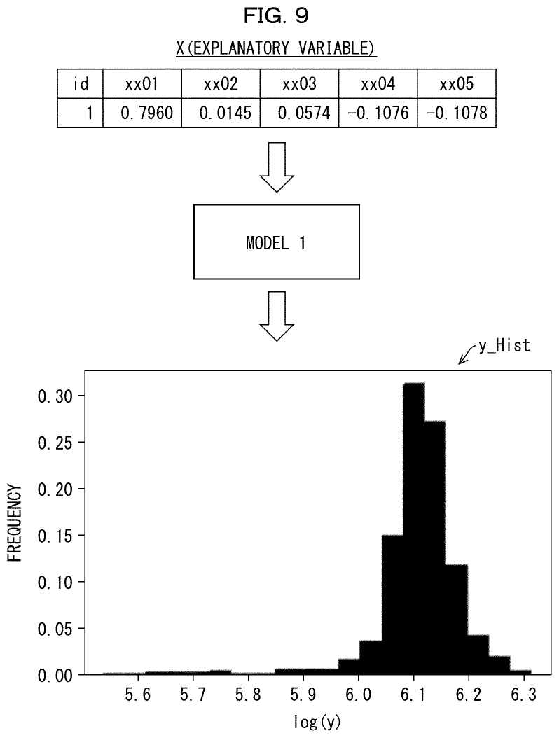

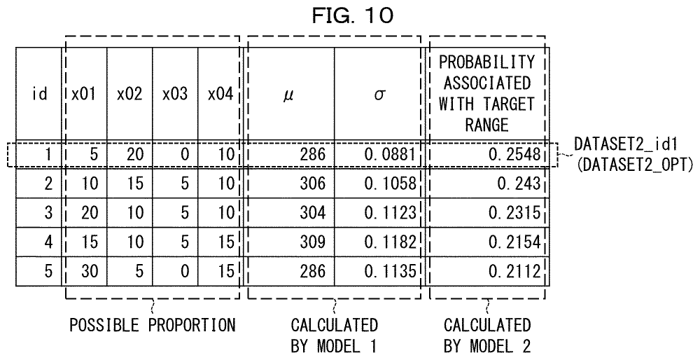

FIG. 9 is a diagram illustrating an example of input and output of MODEL 1. In FIG. 9, the above-described first particle size distribution consequence vector (particle size distribution consequence vector corresponding to id1) is exemplified by X. As illustrated in FIG. 9, it is possible to obtain a histogram (y_Hist) indicating a distribution of y by inputting X to MODEL 1. More specifically, X is inputted to each of the plurality of weak learning devices in MODEL 1, so that y_Hist is obtained by integrating a plurality of objective variables y outputted from the plurality of weak learning devices.

Note in the present example, prior to generation of MODEL 1 by the second algorithm described above, pre-processing of the correct data is carried out. More specifically, in the present example, prior to generation of MODEL 1, logarithmic conversion of the correct data is performed. For this reason, exactly, MODEL 1 is generated as a model that outputs log(y). Accordingly, the horizontal axis of y_Hist in the example of FIG. 9 is log(y). Note that, in the present specification, for simplification, MODEL 1 is described as being a model which outputs y. Further, in the following description, y_Hist is read as a histogram indicating a distribution of y.

MODEL 1 in the present example determines, as a final predicted value (predicted value as a strong learning device), predetermined data based on y_Hist, and outputs the final predicted value. More specifically, MODEL 1 in the present example outputs an average value (μ) of y_Hist as the final predicted value (y). μ is also referred to as an expected value.

According to MODEL 1 generated as the strong learning device, a given input data set 1100 is acquired as the explanatory variable (X) as illustrated in FIG. 2 described above, so that MODEL 1 can output the unknown resin composition characteristic data 1200 as the objective variable (y). For example, it is possible to output, as y, a predicted value of the viscosity corresponding to the first mixing ratio pattern by inputting, as X, the first mixing ratio pattern described above to MODEL 1.

The MODEL 1 can output a variance (σ 2) of y_Hist together with μ. The variance is an example of an index of uncertainty of the predicted value of MODEL 1. Alternatively, MODEL 1 can output a standard deviation (σ) of y_Hist together with μ. The standard deviation is another example of the index of uncertainty of the predicted value of MODEL 1.

### (Operation Example in MODEL 2 Generated by Third Algorithm Execution Section 223)

FIG. 10 is a diagram showing an operation example in MODEL 2 generated by the third algorithm execution section 223. The third algorithm execution section 223 generates MODEL 2 on the basis of MODEL 1 by executing the third algorithm. In other words, the third algorithm execution section 223 determines the above-described function g (an approximate inverse function of the function f) on the basis of the function f determined in advance by the second algorithm execution section 212 (see also FIG. 3 described above).

In the example of FIG. 10, the third algorithm execution section 223 generates MODEL 2 by using grid search as the third algorithm. Inside MODEL 2 generated, two steps (first step and second step) of calculation described below is performed.

First, in the first step, MODEL 2 calculates, with use of MODEL 1, a predicted value (μ) of the viscosity for each of a plurality of possible mixing ratio patterns (combinations of x01 to x04). More specifically, MODEL 2 inputs, to MODEL 1, an explanatory variable (X) corresponding to each mixing ratio pattern. As a result, this MODEL 1 is caused to output μ as an objective variable. In the present example, MODEL 1 further outputs σ in addition to u for each mixing ratio pattern.

Next, in the second step, MODEL 2 outputs, on the basis of μ and σ calculated in the first step, the mixing ratio pattern (X) with which the probability of obtaining given viscosity data that has been inputted to the MODEL 2 is the highest (also see FIG. 3 described above).

In the example of FIG. 10, MODEL 2 predicts the predicted data set 2300 with which the probability of obtaining the given resin composition characteristic data 2200 is the highest. The example of FIG. 10 shows, as an example, a case in which the given resin composition characteristic data 2200 is data indicating a viscosity of “ρ=290 to 310”.

The above numerical value range of μ=290 to 310 is an example of a numerical range that is set on the assumption that the resin composition C is to be used as an adhesive. For example, in a case where the viscosity of the adhesive is too high, it is difficult to form and use the adhesive in a desired shape. On the other hand, in a case where the viscosity of the adhesive is too low, dripping of the adhesive is likely to occur. Therefore, it is considered that a suitable numerical range exists in the viscosity of the adhesive. The numerical value range of μ=290 to 310 is an example of the suitable numerical value range.

In the example of FIG. 10, MODEL 2 selects, as an optimum mixing ratio pattern, the mixing ratio pattern having the highest “probability that μ falls within a target range of 290 to 310” (hereinafter, referred to as “probability associated with the target range”). Then, MODEL 2 outputs the optimum mixing ratio pattern as a prediction result (that is, the predicted data set 2300). In the example of FIG. 10, MODEL 2 calculates the probability associated with the target range for each of all combinations of the plurality of possible mixing ratio patterns. More specifically, in the example of FIG. 10, MODEL 2 calculates the probability associated with the target range on the assumption that u follows a normal distribution.

In the example of FIG. 10, a data set is created for each id. The data set here indicates a corresponding relationship of “each mixing ratio pattern” and “μ and σ” and the “probability associated with the target range”. In the example of FIG. 10, a data set corresponding to the jth id (idj) is referred to as DATASET2_idj.

In the example of FIG. 10, for each of all j, MODEL 2 searches for a data set with which the probability associated with the target range is the highest (hereinafter, referred to as the highest probability data set). In the example of FIG. 10, DATASET2_id1 was found to be the highest probability data set as a result of search by MODEL 2. Note that the first mixing ratio pattern in the example of FIG. 10 is a mixing ratio pattern in which “x00=65, x01=5, x02=20, x03=0, and x04=10”.

MODEL 2 selects the highest probability data set as an optimum data set (DATASET2_OPT). MODEL 2 determines, as the optimum mixing ratio pattern, the mixing ratio pattern corresponding to the optimum data set. In the example of FIG. 10, MODEL 2 selects DATASET2_id1 as the optimum data set. Then, the third algorithm execution section 223 determines, as the optimum mixing ratio pattern, the above-described first mixing ratio pattern corresponding to DATASET2_id1. In the example of FIG. 10, the first mixing ratio pattern is thus outputted as the prediction result.

As described above, the third algorithm execution section 223 generates, by the third algorithm (e.g., grid search), MODEL 2 (a model that predicts the predicted data set 2300 that satisfies the given resin composition characteristic data 2200). However, as would be apparent to a person skilled in the art, a technique for determining the optimum mixing ratio pattern is not limited to the above-described example.

For example, MODEL 2 may search for a mixing ratio pattern corresponding to a data set (hereinafter, referred to as a closest predicted value data set) with which a predicted value (μ) closest to 300 (a median value in a numerical range of the viscosity described above) is obtained, among all of the mixing ratio patterns in the example of FIG. 10. Then, MODEL 2 may determine, as the optimum mixing ratio pattern, the mixing ratio pattern corresponding to the closest predicted value data set.

### (Example of Deriving Recommended Data in Recommended Data Deriving Section 34)

With use of MODEL 2 generated as described above, it is possible to derive, in the recommended data deriving section 34, the recommended data 340 on the basis of the third input data 330 (resin composition required characteristic data) (see also FIG. 4 described above).

For example, the third input data 330 may be data indicating a viscosity of “μ=350 to 370”. In this case, the recommended data deriving section 34 inputs the third input data 330 to MODEL 2, so that the recommended data 340 corresponding to the third input data 330 can be derived. The recommended data 340 in the present example is, for example, an optimum mixing ratio pattern corresponding to the viscosity of μ=350 to 370.

### (Effects)

The model generation device 100 can generate MODEL 2 that predicts the predicted data set 2300 which satisfies the given resin composition characteristic data 2200. In other words, it is possible to generate, as a prediction model, MODEL 2 that outputs the predicted data set 2300 as a prediction result for a required characteristic satisfying condition regarding a resin composition.

Then, the prediction device 300 can predict the required characteristic satisfying condition regarding the resin composition, with use of MODEL 2 that has been generated in advance in the model generation device 100. More specifically, the prediction device 300 can derive the recommended data 340 by inputting resin composition required characteristic data to the MODEL 2. As is apparent from the above descriptions, the recommended data 340 is the prediction result regarding the resin composition required characteristic data.

As described above, the resin composition production system 1 can (i) generate MODEL 2 by machine learning and (ii) predict, with use of this MODEL 2, a required characteristic satisfying condition regarding a resin composition. Therefore, unlike a well-known technique (e.g., the technique of Patent Literature 1), the resin composition production system 1 makes it unnecessary to examine the required characteristic satisfying condition by many trials and errors on an experimental basis. Therefore, the resin composition production system 1 can find the required characteristic satisfying condition regarding the resin composition, more efficiently than a conventional technology.

Therefore, the resin composition production system 1 can develop, for example, a resin composition having a desired required characteristic at a lower cost than a conventional technology. Moreover, the resin composition production system 1 also can realize a novel resin composition, which is expected to contribute to sustainable development goals (SDGs), more easily than a conventional technology.

### ADDITIONAL REMARKS

“Use of machine learning for deriving recommended composition data of a resin composition” is well known per se. However, as far as the inventors have investigated, there is presently no literature that discloses or suggests “generating MODEL 2 (a model that predicts a required characteristic satisfying condition regarding a resin composition C which contains an inorganic filling material A and a resin B) on the basis of a specific combination of pieces of input data disclosed in Embodiment 1”. Therefore, it can be said that the resin composition production system 1 (more specifically, the model generation device 100 and the prediction device 300) is based on a novel technical idea that is sufficiently differentiated from the conventional technology.

### Embodiment 2

Embodiment 1 described, as an example of a first algorithm, weighted average calculation (an algorithm for calculating a weighted average of feature amounts) and principal component analysis (an algorithm for reducing the number of dimensions of the feature amounts). However, as would be apparent to a person skilled in the art, the first algorithm is not limited to those algorithms.

For example, the first algorithm may be an n-th-order moment calculation algorithm (an algorithm for calculating an n-th-order moment of the feature amounts). n is any natural number. Note that a weighted average calculation algorithm in Embodiment 1 corresponds to an n-th-order moment calculation algorithm where n=1. Alternatively, the first algorithm may be an algorithm for calculating a well-known statistics value (e.g.: an average value, a variance, a maximum value, a minimum value, or the like) about the feature amounts.

### Software Implementation Example

The function of the resin composition production system 1 (hereinafter, referred to as “system”) can be realized by a program for causing a computer to function as the system, the program causing the computer to function as each of control blocks (in particular, each section included in the model generation device 100 and the prediction device 300) of the system.

In this case, the system includes, as hardware for executing the program, a computer which includes at least one control device (e.g., processor) and at least one storage device (e.g., memory). By execution of the program with use of the control device and the storage device, each function described in each of the above-described Embodiments is realized.

The program can be stored in at least one computer-readable non-transitory storage medium. The storage medium can be provided in the system, or the storage medium does not need to be provided in the system. In the latter case, the program can be supplied to the system via an arbitrary wired or wireless transmission medium.

Further, one or some or all of respective functions of the control blocks described above can be realized by a logic circuit. For example, an integrated circuit in which a logic circuit that functions as the each control block is formed is also encompassed in the scope of an aspect of the present invention. In addition, it is also possible to realize the each control block by, for example, a quantum computer.

Further, each of processes described in the foregoing embodiments may be executed by artificial intelligence (AI). In this case, the AI may be operated by the control device or may be operated by another device (for example, an edge computer or a cloud server).

Further, an aspect of the present invention encompasses a computer program product that realizes each function of the system. The computer program product causes a program provided via an arbitrary transmission medium to be loaded through at least one computer and causes the computer to execute at least one program command. This causes a processor provided in the at least one computer to execute processing according to the program command, so that each function of the system 10 is realized. The computer program product causes the at least one computer, which has loaded the program, to execute each step of a method for generating a prediction model according to an embodiment of the present invention and each step of a prediction method according to an embodiment of the present invention.

Aspects of the present invention can also be expressed as follows:

A model generation device in accordance with Aspect 1 of the present invention is a model generation device configured to generate a prediction model for predicting at least one selected from the group consisting of the following (i) to (iv), the at least one satisfying a required characteristic of a resin composition which contains at least one inorganic filling material and at least one resin: (i) a characteristic of the inorganic filling material; (ii) a characteristic of the resin; (iii) a mixing ratio of the inorganic filling material; and (iv) a mixing ratio of the resin, the model generation device including:
a first input data acquisition section configured to acquire first input data that is input data including at least one selected from the group consisting of (i) inorganic filling material characteristic input data indicating the characteristic of the inorganic filling material, (ii) resin characteristic input data indicating the characteristic of the resin, (iii) inorganic filling material proportion input data related to the mixing ratio of each of a plurality of the inorganic filling materials in the resin composition obtained by mixing the plurality of the inorganic filling materials in the resin and (iv) resin proportion input data related to the mixing ratio of the resin;
a second input data acquisition section configured to acquire, as second input data that makes a pair with the first input data, resin composition characteristic input data indicating a characteristic of the resin composition;
a first machine learning section configured to generate, on the basis of the first input data and the second input data, a first prediction model that predicts unknown resin composition characteristic data from at least one selected from the group consisting of (i) given inorganic filling material characteristic data, (ii) given resin characteristic data, (iii) given inorganic filling material proportion data and (iv) given resin proportion data; and
a second machine learning section configured to generate, on the basis of the first prediction model, a second prediction model that predicts at least one selected from the group consisting of (i) predicted inorganic filling material characteristic data, (ii) predicted resin characteristic data, (iii) predicted inorganic filling material proportion data and (iv) predicted resin proportion data, the at least one satisfying given resin composition characteristic data.

A model generation device in accordance with Aspect 2 of the present invention may be configured such that in the above-described Aspect 1, the first machine learning section includes: a first algorithm execution section configured to execute a first algorithm for deriving an explanatory variable corresponding to data that indicates the characteristic of the resin composition and that is obtained from the second input data; and a second algorithm execution section configured to execute a second algorithm for generating the first prediction model on the basis of the explanatory variable and the second input data.

A model generation device in accordance with Aspect 3 of the present invention may be configured such that in the above-described Aspect 2, the second algorithm is at least one selected from the group consisting of Gaussian process regression, a support-vector machine, linear regression, a decision tree, random forest, a neural network and a gradient boosting decision tree.

A model generation device in accordance with Aspect 4 of the present invention may be configured such that in any one of the above-described Aspects 1 to 3, the second machine learning section has a third algorithm execution section configured to execute a third algorithm for generating the second prediction model on the basis of the first prediction model.

A model generation device in accordance with Aspect 5 of the present invention may be configured such that in the above-described Aspect 4, the third algorithm is at least one selected from the group consisting of genetic algorithm, gradient descent, grid search, and Bayesian optimization.

A model generation device in accordance with Aspect 6 of the present invention may be configured such that in any one of the above-described Aspects 1 to 5, the inorganic filling material characteristic input data indicates, as the characteristic of the inorganic filling material, at least one selected from the group consisting of
composition formula, crystallinity, specific gravity, bulk specific gravity, particle size distribution, specific surface area, pore volume, zeta potential, specific electric conductivity, dielectric constant, dielectric dissipation factor, refractive index, specific heat, thermal conductivity, linear expansion coefficient, crushing strength, sphericity, aspect ratio, moisture content, carbon content, nitrogen content, surface functional group species, surface functional group content, light absorption wavelength, light absorbance, M-value and solubility parameter of the inorganic filling material.

A model generation device in accordance with Aspect 7 of the present invention may be configured such that in any one of the above-described Aspects 1 to 6, the resin characteristic input data indicates, as the characteristic of the resin, at least one selected from the group consisting of
composition formula, polymerization degree, molecular weight distribution, stereoregularity, reactive functional group species, reactive functional group point, glass transition content, viscosity, melting temperature, crystallinity, elastic modulus, yield stress, breaking strength, fracture toughness, light absorption wavelength, light absorbance, specific gravity, refractive index, specific electric conductivity, dielectric constant, dielectric dissipation factor, specific heat, thermal conductivity, moisture content and solubility parameter of the resin.

A model generation device in accordance with Aspect 8 of the present invention may be configured such that in any one of the above-described Aspects 1 to 7, the resin composition characteristic input data indicates, as the characteristic of the resin composition, at least one selected from the group consisting of
viscosity, flowability, moldability, adhesiveness, transparency, color tone, strength, water absorption rate, linear expansion coefficient, elastic modulus, yield stress, breaking strength, fracture toughness, specific electric conductivity, dielectric constant, dielectric dissipation factor, thermal conductivity and stability of the resin composition.

A prediction device in accordance with Aspect 9 of the present invention is a prediction device configured to predict at least one selected from the group consisting of the following (i) to (iv), the at least one satisfying a required characteristic of a resin composition which contains at least one inorganic filling material and at least one resin: (i) a characteristic of the inorganic filling material; (ii) a characteristic of the resin; (iii) a mixing ratio of the inorganic filling material; and (iv) a mixing ratio of the resin, wherein:
first input data is acquired in advance, the first input data being input data including at least one selected from the group consisting of (i) inorganic filling material characteristic input data indicating the characteristic of the inorganic filling material, (ii) resin characteristic input data indicating the characteristic of the resin, (iii) inorganic filling material proportion input data related to the mixing ratio of each of a plurality of the inorganic filling materials in the resin composition obtained by mixing the plurality of the inorganic filling materials in the resin and (iv) resin proportion input data related to the mixing ratio of the resin;
resin composition characteristic input data is acquired in advance, as second input data that makes a pair with the first input data, the resin composition characteristic input data indicating a characteristic of the resin composition;
a first prediction model is generated in advance, on the basis of the first input data and the second input data, the first prediction model predicting unknown resin composition characteristic data from at least one selected from the group consisting of (i) given inorganic filling material characteristic data, (ii) given resin characteristic data, (iii) given inorganic filling material proportion data and (iv) given resin proportion data;
a second prediction model is generated in advance, on the basis of the first prediction model, the second prediction model predicting at least one selected from the group consisting of (i) predicted inorganic filling material characteristic data, (ii) predicted resin characteristic data, (iii) predicted inorganic filling material proportion data and (iv) predicted resin proportion data, the at least one satisfying given resin composition characteristic data; and
the prediction device includes:
   a third input data acquisition section configured to acquire, as third input data, resin composition required characteristic data indicating the required characteristic of the resin composition; and
   a recommended data deriving section configured to derive recommended data by inputting the resin composition required characteristic data to the second prediction model, the recommended data including at least one selected from the group consisting of (i) recommended inorganic filling material characteristic data indicating the characteristic of the inorganic filling material that satisfies the resin composition required characteristic data, (ii) recommended resin characteristic data indicating the characteristic of the resin that satisfies the resin composition required characteristic data, (iii) recommended inorganic filling material proportion data indicating a proportion of the inorganic filling material that satisfies the resin composition required characteristic data and (iv) recommended resin proportion data indicating a proportion of the resin that satisfies the resin composition required characteristic data.

A prediction device in accordance with Aspect 10 of the present invention may be configured such that in the above-described Aspect 9, the resin composition required characteristic data indicates, as the required characteristic of the resin composition, at least one selected from the group consisting of
viscosity, flowability, moldability, adhesiveness, transparency, color tone, strength, water absorption rate, linear expansion coefficient, elastic modulus, yield stress, breaking strength, fracture toughness, specific electric conductivity, dielectric constant, dielectric dissipation factor, thermal conductivity and stability of the resin composition.

A model generation method in accordance with Aspect 11 of the present invention is a method for generating a prediction model for predicting at least one selected from the group consisting of the following (i) to (iv), the at least one satisfying a required characteristic of a resin composition which contains at least one inorganic filling material and at least one resin: (i) a characteristic of the inorganic filling material; (ii) a characteristic of the resin; (iii) a mixing ratio of the inorganic filling material; and (iv) a mixing ratio of the resin,
the method including:
a first input data acquisition step of acquiring first input data that is input data including at least one selected from the group consisting of (i) inorganic filling material characteristic input data indicating the characteristic of the inorganic filling material, (ii) resin characteristic input data indicating the characteristic of the resin, (iii) inorganic filling material proportion input data related to the mixing ratio of each of a plurality of the inorganic filling materials in the resin composition obtained by mixing the plurality of the inorganic filling materials in the resin and (iv) resin proportion input data related to the mixing ratio of the resin;
a second input data acquisition step of acquiring, as second input data that makes a pair with the first input data, resin composition characteristic input data indicating a characteristic of the resin composition;
a first machine learning step of generating, on the basis of the first input data and the second input data, a first prediction model that predicts unknown resin composition characteristic data from at least one selected from the group consisting of (i) given inorganic filling material characteristic data, (ii) given resin characteristic data, (iii) given inorganic filling material proportion data and (iv) given resin proportion data; and
a second machine learning step of generating, on the basis of the first prediction model, a second prediction model that predicts at least one selected from the group consisting of (i) predicted inorganic filling material characteristic data, (ii) predicted resin characteristic data, (iii) predicted inorganic filling material proportion data and (iv) predicted resin proportion data, the at least one satisfying given resin composition characteristic data.

A model generation method in accordance with Aspect 12 of the present invention is a method for predicting at least one selected from the group consisting of the following (i) to (iv), the at least one satisfying a required characteristic of a resin composition which contains at least one inorganic filling material and at least one resin: (i) a characteristic of the inorganic filling material; (ii) a characteristic of the resin; (iii) a mixing ratio of the inorganic filling material; and (iv) a mixing ratio of the resin, wherein:
first input data is acquired in advance, the first input data being input data including at least one selected from the group consisting of (i) inorganic filling material characteristic input data indicating the characteristic of the inorganic filling material, (ii) resin characteristic input data indicating the characteristic of the resin, (iii) inorganic filling material proportion input data related to the mixing ratio of each of a plurality of the inorganic filling materials in the resin composition obtained by mixing the plurality of the inorganic filling materials in the resin and (iv) resin proportion input data related to the mixing ratio of the resin;
resin composition characteristic input data is acquired in advance, as second input data that makes a pair with the first input data, the resin composition characteristic input data indicating a characteristic of the resin composition;
a first prediction model is generated in advance, on the basis of the first input data and the second input data, the first prediction model predicting unknown resin composition characteristic data from at least one selected from the group consisting of (i) given inorganic filling material characteristic data, (ii) given resin characteristic data, (iii) given inorganic filling material proportion data and (iv) given resin proportion data;
a second prediction model is generated in advance, on the basis of the first prediction model, the second prediction model predicting at least one selected from the group consisting of (i) predicted inorganic filling material characteristic data, (ii) predicted resin characteristic data, (iii) predicted inorganic filling material proportion data and (iv) predicted resin proportion data, the at least one satisfying given resin composition characteristic data; and
the method includes:
   a third input data acquisition step of acquiring, as third input data, resin composition required characteristic data indicating the required characteristic of the resin composition; and
   a recommended data deriving step of deriving recommended data by inputting the resin composition required characteristic data to the second prediction model, the recommended data including at least one selected from the group consisting of (i) recommended inorganic filling material characteristic data indicating the characteristic of the inorganic filling material that data, (ii) recommended resin characteristic data indicating the characteristic of the resin that satisfies the resin composition required characteristic data, (iii) recommended inorganic filling material proportion data indicating a proportion of the inorganic filling material that satisfies the resin composition required characteristic data and (iv) recommended resin proportion data indicating a proportion of the resin that satisfies the resin composition required characteristic data.

A resin composition production system in accordance with Aspect 13 of the present invention is a resin composition production system including:
a model generation device configured to generate a prediction model for predicting at least one selected from the group consisting of the following (i) to (iv), the at least one satisfying a required characteristic of a resin composition which contains at least one inorganic filling material and at least one resin: (i) a characteristic of the inorganic filling material; (ii) a characteristic of the resin; (iii) a mixing ratio of the inorganic filling material; and (iv) a mixing ratio of the resin; and
a prediction device configured to predict, with use of the prediction model generated by the model generation device, at least one selected from the group consisting of the following (i) to (iv), the at least one satisfying the required characteristic of the resin composition: (i) the characteristic of the inorganic filling material; (ii) the characteristic of the resin; (iii) the mixing ratio of the inorganic filling material; and (iv) the mixing ratio of the resin,
the model generation device including:
   a first input data acquisition section configured to acquire first input data that is input data including at least one selected from the group consisting of (i) inorganic filling material characteristic input data indicating the characteristic of the inorganic filling material, (ii) resin characteristic input data indicating the characteristic of the resin, (iii) inorganic filling material proportion input data related to the mixing ratio of each of a plurality of the inorganic filling materials in the resin composition obtained by mixing the plurality of the inorganic filling materials in the resin and (iv) resin proportion input data related to the mixing ratio of the resin;
   a second input data acquisition section configured to acquire, as second input data that makes a pair with the first input data, resin composition characteristic input data indicating a characteristic of the resin composition;
   a first machine learning section configured to generate, on the basis of the first input data and the second input data, a first prediction model that predicts unknown resin composition characteristic data from at least one selected from the group consisting of (i) given inorganic filling material characteristic data, (ii) given resin characteristic data, (iii) given inorganic filling material proportion data and (iv) given resin proportion data; and
   a second machine learning section configured to generate, on the basis of the first prediction model, a second prediction model that predicts at least one selected from the group consisting of (i) predicted inorganic filling material characteristic data, (ii) predicted resin characteristic data, (iii) predicted inorganic filling material proportion data and (iv) predicted resin proportion data, the at least one satisfying given resin composition characteristic data,
the prediction device including:
   a third input data acquisition section configured to acquire, as third input data, resin composition required characteristic data indicating the required characteristic of the resin composition; and
   a recommended data deriving section configured to derive recommended data by inputting the resin composition required characteristic data to the second prediction model, the recommended data including at least one selected from the group consisting of (i) recommended inorganic filling material characteristic data indicating the characteristic of the inorganic filling material that satisfies the resin composition required characteristic data, (ii) recommended resin characteristic data indicating the characteristic of the resin that satisfies the resin composition required characteristic data, (iii) recommended inorganic filling material proportion data indicating a proportion of the inorganic filling material that satisfies the resin composition required characteristic data and (iv) recommended resin proportion data indicating a proportion of the resin that satisfies the resin composition required characteristic data.

### ADDITIONAL REMARK

An aspect of the present invention is not limited to any of the embodiments described above, but may be altered in various ways by a skilled person within the scope of the claims. Specifically, any embodiment based on a proper combination of technical means disclosed in different embodiments is also encompassed in the technical scope of an aspect of the present invention.

### REFERENCE SIGNS LIST

1 resin composition production system
11 first input data acquisition section
12 second input data acquisition section
21 first machine learning section
22 second machine learning section
33 third input data acquisition section
34 recommended data deriving section
35 output section
100 model generation device
110 first input data
111 inorganic filling material characteristic input data
112 resin characteristic input data
113 inorganic filling material proportion input data
114 resin proportion input data
120 second input data (resin composition characteristic input data)
211 first algorithm execution section
212 second algorithm execution section
223 third algorithm execution section
300 prediction device
330 third input data (resin composition required characteristic data)
340 recommended data
341 recommended inorganic filling material characteristic data
342 recommended resin characteristic data
343 recommended inorganic filling material proportion data
344 recommended resin proportion data
1100 given input data set
1110 given inorganic filling material characteristic data
1120 given resin characteristic data
1130 given inorganic filling material proportion data
1140 given resin proportion data
1200 unknown resin composition characteristic data
2200 given resin composition characteristic data
2300 predicted data set
2310 predicted inorganic filling material characteristic data
2330 predicted inorganic filling material proportion data
2340 predicted resin proportion data
MODEL 1 first prediction model
MODEL 2 second prediction model
x explanatory variable
y objective variable

1. A model generation device configured to generate a prediction model for predicting at least one selected from the group consisting of the following (i) to (iv), the at least one satisfying a required characteristic of a resin composition which contains at least one inorganic filling material and at least one resin: (i) a characteristic of the inorganic filling material: (ii) a characteristic of the resin; (iii) a mixing ratio of the inorganic filling material; and (iv) a mixing ratio of the resin,
the model generation device comprising:
a first input data acquisition section configured to acquire first input data that is input data including at least one selected from the group consisting of (i) inorganic filling material characteristic input data indicating the characteristic of the inorganic filling material, (ii) resin characteristic input data indicating the characteristic of the resin, (iii) inorganic filling material proportion input data related to the mixing ratio of each of a plurality of the inorganic filling materials in the resin composition obtained by mixing the plurality of the inorganic filling materials in the resin and (iv) resin proportion input data related to the mixing ratio of the resin;
a second input data acquisition section configured to acquire, as second input data that makes a pair with the first input data, resin composition characteristic input data indicating a characteristic of the resin composition;
a first machine learning section configured to generate, on the basis of the first input data and the second input data, a first prediction model that predicts unknown resin composition characteristic data from at least one selected from the group consisting of (i) given inorganic filling material characteristic data, (ii) given resin characteristic data, (iii) given inorganic filling material proportion data and (iv) given resin proportion data; and
a second machine learning section configured to generate, on the basis of the first prediction model, a second prediction model that predicts at least one selected from the group consisting of (i) predicted inorganic filling material characteristic data, (ii) predicted resin characteristic data, (iii) predicted inorganic filling material proportion data and (iv) predicted resin proportion data, the at least one satisfying given resin composition characteristic data.

2. The model generation device according to claim 1, wherein the first machine learning section includes:
a first algorithm execution section configured to execute a first algorithm for deriving an explanatory variable corresponding to data that indicates the characteristic of the resin composition and that is obtained from the second input data; and
a second algorithm execution section configured to execute a second algorithm for generating the first prediction model on the basis of the explanatory variable and the second input data.

3. The model generation device according to claim 2, wherein the second algorithm is at least one selected from the group consisting of
Gaussian process regression, a support-vector machine, linear regression, a decision tree, random forest, a neural network and a gradient boosting decision tree.

4. The model generation device according to claim 1, wherein the second machine learning section has a third algorithm execution section configured to execute a third algorithm for generating the second prediction model on the basis of the first prediction model.

5. The model generation device according to claim 4, wherein the third algorithm is at least one selected from the group consisting of
genetic algorithm, gradient descent, grid search, and Bayesian optimization.

6. The model generation device according to claim 1, wherein the inorganic filling material characteristic input data indicates, as the characteristic of the inorganic filling material, at least one selected from the group consisting of
composition formula, crystallinity, specific gravity, bulk specific gravity, particle size distribution, specific surface area, pore volume, zeta potential, specific electric conductivity, dielectric constant, dielectric dissipation factor, refractive index, specific heat, thermal conductivity, linear expansion coefficient, crushing strength, sphericity, aspect ratio, moisture content, carbon content, nitrogen content, surface functional group species, surface functional group content, light absorption wavelength, light absorbance, M-value and solubility parameter of the inorganic filling material.

7. The model generation device according to claim 1, wherein the resin characteristic input data indicates, as the characteristic of the resin, at least one selected from the group consisting of
composition formula, polymerization degree, molecular weight distribution, stereoregularity, reactive functional group species, reactive functional group content, viscosity, melting point, glass transition temperature, crystallinity, elastic modulus, yield stress, breaking strength, fracture toughness, light absorption wavelength, light absorbance, specific gravity, refractive index, specific electric conductivity, dielectric constant, dielectric dissipation factor, specific heat, thermal conductivity, moisture content and solubility parameter of the resin.

8. The model generation device according to claim 1, wherein the resin composition characteristic input data indicates, as the characteristic of the resin composition, at least one selected from the group consisting of
viscosity, flowability, moldability, adhesiveness, transparency, color tone, strength, water absorption rate, linear expansion coefficient, elastic modulus, yield stress, breaking strength, fracture toughness, specific electric conductivity, dielectric constant, dielectric dissipation factor, thermal conductivity and stability of the resin composition.

9. A prediction device configured to predict at least one selected from the group consisting of the following (i) to (iv), the at least one satisfying a required characteristic of a resin composition which contains at least one inorganic filling material and at least one resin: (i) a characteristic of the inorganic filling material: (ii) a characteristic of the resin: (iii) a mixing ratio of the inorganic filling material; and (iv) a mixing ratio of the resin, wherein:
first input data is acquired in advance, the first input data being input data including at least one selected from the group consisting of (i) inorganic filling material characteristic input data indicating the characteristic of the inorganic filling material, (ii) resin characteristic input data indicating the characteristic of the resin, (iii) inorganic filling material proportion input data related to the mixing ratio of each of a plurality of the inorganic filling materials in the resin composition obtained by mixing the plurality of the inorganic filling materials in the resin and (iv) resin proportion input data related to the mixing ratio of the resin;
resin composition characteristic input data is acquired in advance, as second input data that makes a pair with the first input data, the resin composition characteristic input data indicating a characteristic of the resin composition;
a first prediction model is generated in advance, on the basis of the first input data and the second input data, the first prediction model predicting unknown resin composition characteristic data from at least one selected from the group consisting of (i) given inorganic filling material characteristic data, (ii) given resin characteristic data, (iii) given inorganic filling material proportion data and (iv) given resin proportion data;
a second prediction model is generated in advance, on the basis of the first prediction model, the second prediction model predicting at least one selected from the group consisting of (i) predicted inorganic filling material characteristic data, (ii) predicted resin characteristic data, (iii) predicted inorganic filling material proportion data and (iv) predicted resin proportion data, the at least one satisfying given resin composition characteristic data; and
the prediction device includes:
a third input data acquisition section configured to acquire, as third input data, resin composition required characteristic data indicating the required characteristic of the resin composition; and
a recommended data deriving section configured to derive recommended data by inputting the resin composition required characteristic data to the second prediction model, the recommended data including at least one selected from the group consisting of (i) recommended inorganic filling material characteristic data indicating the characteristic of the inorganic filling material that satisfies the resin composition required characteristic data, (ii) recommended resin characteristic data indicating the characteristic of the resin that satisfies the resin composition required characteristic data, (iii) recommended inorganic filling material proportion data indicating a proportion of the inorganic filling material that satisfies the resin composition required characteristic data and (iv) recommended resin proportion data indicating a proportion of the resin that satisfies the resin composition required characteristic data.

10. The prediction device according to claim 9, wherein the resin composition required characteristic data indicates, as the required characteristic of the resin composition, at least one selected from the group consisting of
viscosity, flowability, moldability, adhesiveness, transparency, color tone, strength, water absorption rate, linear expansion coefficient, elastic modulus, yield stress, breaking strength, fracture toughness, specific electric conductivity, dielectric constant, dielectric dissipation factor, thermal conductivity and stability of the resin composition.

11. A method for generating a prediction model for predicting at least one selected from the group consisting of the following (i) to (iv), the at least one satisfying a required characteristic of a resin composition which contains at least one inorganic filling material and at least one resin: (i) a characteristic of the inorganic filling material; (ii) a characteristic of the resin: (iii) a mixing ratio of the inorganic filling material; and (iv) a mixing ratio of the resin,
the method comprising:
a first input data acquisition step of acquiring first input data that is input data including at least one selected from the group consisting of (i) inorganic filling material characteristic input data indicating the characteristic of the inorganic filling material, (ii) resin characteristic input data indicating the characteristic of the resin, (iii) inorganic filling material proportion input data related to the mixing ratio of each of a plurality of the inorganic filling materials in the resin composition obtained by mixing the plurality of the inorganic filling materials in the resin and (iv) resin proportion input data related to the mixing ratio of the resin;
a second input data acquisition step of acquiring, as second input data that makes a pair with the first input data, resin composition characteristic input data indicating a characteristic of the resin composition;
a first machine learning step of generating, on the basis of the first input data and the second input data, a first prediction model that predicts unknown resin composition characteristic data from at least one selected from the group consisting of (i) given inorganic filling material characteristic data, (ii) given resin characteristic data, (iii) given inorganic filling material proportion data and (iv) given resin proportion data; and
a second machine learning step of generating, on the basis of the first prediction model, a second prediction model that predicts at least one selected from the group consisting of (i) predicted inorganic filling material characteristic data, (ii) predicted resin characteristic data, (iii) predicted inorganic filling material proportion data and (iv) predicted resin proportion data, the at least one satisfying given resin composition characteristic data.

12. A method for predicting at least one selected from the group consisting of the following (i) to (iv), the at least one satisfying a required characteristic of a resin composition which contains at least one inorganic filling material and at least one resin: (i) a characteristic of the inorganic filling material: (ii) a characteristic of the resin; (iii) a mixing ratio of the inorganic filling material; and (iv) a mixing ratio of the resin, wherein:
first input data is acquired in advance, the first input data being input data including at least one selected from the group consisting of (i) inorganic filling material characteristic input data indicating the characteristic of the inorganic filling material, (ii) resin characteristic input data indicating the characteristic of the resin, (iii) inorganic filling material proportion input data related to the mixing ratio of each of a plurality of the inorganic filling materials in the resin composition obtained by mixing the plurality of the inorganic filling materials in the resin and (iv) resin proportion input data related to the mixing ratio of the resin;
resin composition characteristic input data is acquired in advance, as second input data that makes a pair with the first input data, the resin composition characteristic input data indicating a characteristic of the resin composition;
a first prediction model is generated in advance, on the basis of the first input data and the second input data, the first prediction model predicting unknown resin composition characteristic data from at least one selected from the group consisting of (i) given inorganic filling material characteristic data, (ii) given resin characteristic data, (iii) given inorganic filling material proportion data and (iv) given resin proportion data;
a second prediction model is generated in advance, on the basis of the first prediction model, the second prediction model predicting at least one selected from the group consisting of (i) predicted inorganic filling material characteristic data, (ii) predicted resin characteristic data, (iii) predicted inorganic filling material proportion data and (iv) predicted resin proportion data, the at least one satisfying given resin composition characteristic data; and
the method includes:
a third input data acquisition step of acquiring, as third input data, resin composition required characteristic data indicating the required characteristic of the resin composition; and
a recommended data deriving step of deriving recommended data by inputting the resin composition required characteristic data to the second prediction model, the recommended data including at least one selected from the group consisting of (i) recommended inorganic filling material characteristic data indicating the characteristic of the inorganic filling material that satisfies the resin composition required characteristic data, (ii) recommended resin characteristic data indicating the characteristic of the resin that satisfies the resin composition required characteristic data, (iii) recommended inorganic filling material proportion data indicating a proportion of the inorganic filling material that satisfies the resin composition required characteristic data and (iv) recommended resin proportion data indicating a proportion of the resin that satisfies the resin composition required characteristic data.

13. A resin composition production system comprising:
a model generation device configured to generate a prediction model for predicting at least one selected from the group consisting of the following (i) to (iv), the at least one satisfying a required characteristic of a resin composition which contains at least one inorganic filling material and at least one resin: (i) a characteristic of the inorganic filling material: (ii) a characteristic of the resin: (iii) a mixing ratio of the inorganic filling material; and (iv) a mixing ratio of the resin; and
a prediction device configured to predict, with use of the prediction model generated by the model generation device, at least one selected from the group consisting of the following (i) to (iv), the at least one satisfying the required characteristic of the resin composition: (i) the characteristic of the inorganic filling material: (ii) the characteristic of the resin: (iii) the mixing ratio of the inorganic filling material; and (iv) the mixing ratio of the resin,
the model generation device including:
a first input data acquisition section configured to acquire first input data that is input data including at least one selected from the group consisting of (i) inorganic filling material characteristic input data indicating the characteristic of the inorganic filling material, (ii) resin characteristic input data indicating the characteristic of the resin, (iii) inorganic filling material proportion input data related to the mixing ratio of each of a plurality of the inorganic filling materials in the resin composition obtained by mixing the plurality of the inorganic filling materials in the resin and (iv) resin proportion input data related to the mixing ratio of the resin;
a second input data acquisition section configured to acquire, as second input data that makes a pair with the first input data, resin composition characteristic input data indicating a characteristic of the resin composition;
a first machine learning section configured to generate, on the basis of the first input data and the second input data, a first prediction model that predicts unknown resin composition characteristic data from at least one selected from the group consisting of (i) given inorganic filling material characteristic data, (ii) given resin characteristic data, (iii) given inorganic filling material proportion data and (iv) given resin proportion data; and
a second machine learning section configured to generate, on the basis of the first prediction model, a second prediction model that predicts at least one selected from the group consisting of (i) predicted inorganic filling material characteristic data, (ii) predicted resin characteristic data, (iii) predicted inorganic filling material proportion data and (iv) predicted resin proportion data, the at least one satisfying given resin composition characteristic data,
the prediction device including:
a third input data acquisition section configured to acquire, as third input data, resin composition required characteristic data indicating the required characteristic of the resin composition; and
a recommended data deriving section configured to derive recommended data by inputting the resin composition required characteristic data to the second prediction model, the recommended data including at least one selected from the group consisting of (i) recommended inorganic filling material characteristic data indicating the characteristic of the inorganic filling material that satisfies the resin composition required characteristic data, (ii) recommended resin characteristic data indicating the characteristic of the resin that satisfies the resin composition required characteristic data, (iii) recommended inorganic filling material proportion data indicating a proportion of the inorganic filling material that satisfies the resin composition required characteristic data and (iv) recommended resin proportion data indicating a proportion of the resin that satisfies the resin composition required characteristic data.
